(19) United States
(12) Patent Application Publication
(10) Pub. No.: US 2024/0382224 A1
(43) Pub. Date: Nov. 21, 2024

(54) SURGICAL DEVICES, SYSTEMS, AND METHODS INCLUDING ADAPTIVE CONTROL

(71) Applicant: Mazor Robotics Ltd., Caesarea (IL)

(72) Inventors: Michael Vu, Grand Prairie, TX (US); Milton F. Barnes, Grand Prairie, TX (US); Jaffar Hleihil, Zichron Yaakov (IL); Aayush Malla, Fort Worth, TX (US); Andrew J. Wald, Fort Worth, TX (US); Mahin Maharjan, Arlington, TX (US); Saideep Nakka, Northlake, TX (US); Haoran Li, Flower Mound, TX (US); Sophie A. Pervere, Dallas, TX (US); Bret R. Hauser, Fort Worth, TX (US); John W. Kulas, Euless, TX (US)

(21) Appl. No.: 18/662,123

(22) Filed: May 13, 2024

Related U.S. Application Data
(60) Provisional application No. 63/466,628, filed on May 15, 2023.

(51) Int. Cl. A61B 17/32; A61B 17/00; A61B 90/00; G16H 40/63

(52) U.S. Cl. CPC A61B 17/32002; A61B 90/06; G16H 40/63; A61B 2017/00084; A61B 2017/00199; A61B 2017/00398; A61B 2017/0046; A61B 2017/320028; A61B 2090/066

(57) ABSTRACT

A surgical system having adaptive control includes a surgical cutting device, at least one sensor, and a controller. The surgical cutting device includes a cutting tool and a motor configured to drive movement of the cutting tool. The at least one sensor is configured to produce sensor data indicative of at least one property of the surgical cutting device during use. The controller is configured to receive the sensor data and determine a performance condition of the surgical cutting device based at least on the sensor data. The controller is further configured, where the determined performance condition is an adverse performance condition, to at least one of: adjust settings of the surgical cutting device or recommend a change relating to use of the surgical cutting device.

### CROSS-REFERENCE TO RELATED APPLICATION

This application claims the benefit of and priority to U.S. Provisional Patent Application No. 63/466,628 filed May 15, 2023, the entire disclosure of which is incorporated by reference herein.

### FIELD

The present disclosure relates to surgical devices, systems, and methods and, more particularly, to surgical devices, systems, and methods including adaptive control.

### BACKGROUND

Powered surgical cutting devices and systems are utilized in a wide variety of surgical procedures to perform various different surgical cutting functions including, for example, drilling, tapping, resection, dissection, debridement, shaving, sawing, pulverizing, and/or shaping of anatomical tissue including bone.

Many of such powered surgical cutting devices and systems are precisely designed to ensure the device functions safely and effectively. However, regardless of the precision of design, situations may be encountered during use that result in erratic performance, loss of stability, reduced efficiency or effectiveness, and/or other adverse conditions.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent (or greater, depending upon industry standards). To the extent consistent, any of the aspects described herein may be used in conjunction with any or all of the other aspects described herein.

Provided in accordance with aspects of the present disclosure is a surgical system having adaptive control. The surgical system includes a surgical cutting device, at least one sensor, and a controller. The surgical cutting device includes a cutting tool and a motor configured to drive movement of the cutting tool. The at least one sensor is configured to produce sensor data indicative of at least one property of the surgical cutting device during use. The controller is configured to receive the sensor data and determine a performance condition of the surgical cutting device based at least on the sensor data. The controller is further configured, where the determined performance condition is an adverse performance condition, to at least one of: adjust settings of the surgical cutting device or recommend a change relating to use of the surgical cutting device.

In an aspect of the present disclosure, the controller is further configured to receive other data and to determine the performance condition based at least on the sensor data and the other data. The other data may include, in aspects, identifying data, patient data, and/or procedure data.

In another aspect of the present disclosure, the determined performance condition includes at least one of a stability condition or an efficiency condition. Additionally or alternatively, the determined performance condition may be a binary determination or a scaled determination.

In still another aspect of the present disclosure, the controller is configured, where the determined performance condition is an adverse performance condition, to adjust settings of the surgical cutting device by adjusting at least one of: a speed of the motor; a torque of the motor; an operating mode; or a performance impacting component such as a dampening component (e.g., a position, property, etc. of the dampening component or other performance impacting component) of the surgical cutting device.

In yet another aspect of the present disclosure, the controller is configured, where the determined performance condition is an adverse performance condition, to recommend a change relating to use of the surgical cutting device by recommending: a change in ergonomic position, a change in technique, a manual change to a performance impacting component such as a dampening component (e.g., a position, property, etc. of the dampening component or other performance impacting component) of the surgical cutting device; or changing to a different surgical cutting device or portion thereof.

In still yet another aspect of the present disclosure, the at least one sensor includes at least one of: a vibration sensor, a position sensor, an acceleration sensor, an optical sensor, an audio sensor, a force sensor, a temperature sensor, and/or a motor electrical property sensor.

In another aspect of the present disclosure, the surgical cutting device further includes a handle housing the motor therein and a shaft assembly coupled to the handle and including an outer sleeve. The cutting tool, in such aspects, extends through the outer sleeve of the shaft assembly. In these aspects, the at least one sensor may be disposed on or within at least one of: the handle, the outer sleeve, or the cutting tool.

In yet another aspect of the present disclosure, the controller is configured to operate in near real time, e.g., to determine the performance condition and, where the determined performance condition is an adverse performance condition, at least one of: adjust or recommend.

In still another aspect of the present disclosure, the surgical system includes a console configured to supply power and control signals to the surgical cutting device. In such aspects, the controller is disposed within the console.

In still yet another aspect of the present disclosure, the controller is configured to implement at least one machine learning algorithm to determine the performance condition.

A method of adaptive control of a surgical system provided in accordance with the present disclosure includes: driving a motor to move a cutting tool of a surgical cutting device to cut tissue; monitoring, during the cutting of the tissue, sensor data indicative of at least one property of the surgical cutting device; determining a performance condition of the surgical cutting device based at least on the sensor data; and where the determined performance condition is an adverse performance condition, at least one of: adjusting settings of the surgical cutting device or recommending a change relating to use of the surgical cutting device.

In an aspect of the present disclosure, the method further includes receiving other data including at least one of: identifying data, patient data, or procedure data. The performance condition, in such aspects, is determined based at least on the sensor data and the other data.

In another aspect of the present disclosure, the determined performance condition includes at least one of a stability condition or an efficiency condition.

In still another aspect of the present disclosure, where the determined performance condition is an adverse performance condition, the settings of the surgical cutting device are adjusted by adjusting at least one of: a speed of the motor; a torque of the motor; an operating mode; or a performance impacting component such as a dampening component (e.g., a position, property, etc. of the dampening component or other performance impacting component) of the surgical cutting device.

In yet another aspect of the present disclosure, where the determined performance condition is an adverse performance condition, a change relating to use of the surgical cutting device is recommended by recommending: a change in ergonomic position, a change in technique, a manual change to a performance impacting component such as a dampening component (e.g., a position, property, etc. of the dampening component or other performance impacting component) of the surgical cutting device; or changing to a different surgical cutting device or portion thereof.

In still yet another aspect of the present disclosure, the sensor data includes at least one of: vibration data, position data, acceleration data, optical data, audio data, force data, temperature data, and/or motor electrical property data.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a surgical system in accordance with the present disclosure including a console and a powered surgical cutting device;

FIG. 2 illustrates various different rotational cutting tips configured for use with the powered surgical cutting device of FIG. 1;

FIGS. 3A-3C are perspective views of various different surgical saw-type powered surgical cutting devices configured for use with the surgical system of FIG. 1;

FIG. 4 is a side view of the powered surgical cutting device of FIG. 1 including a plurality of sensors disposed at various different locations in accordance with the present disclosure;

FIG. 5 is a longitudinal, cross-sectional view of the powered surgical cutting device of FIG. 1 including a plurality of sensors disposed at various different locations in accordance with the present disclosure;

FIG. 6 is a block diagram of a controller of the console of FIG. 1;

FIG. 7 is a logic diagram of an algorithm in accordance with the present disclosure;

FIG. 8 is a logic diagram of a machine learning algorithm in accordance with the present disclosure;

FIG. 9 is a flow diagram of a method in accordance with the present disclosure;

FIGS. 10A and 10B are side, partial cross-sectional views of a distal portion of the powered surgical cutting device of FIG. 1 in use cutting tissue in initial and adjusted configurations, respectively; and

FIGS. 11A and 11B are front views of the console of FIG. 1 displaying initial and adjusted settings, respectively.

### DETAILED DESCRIPTION

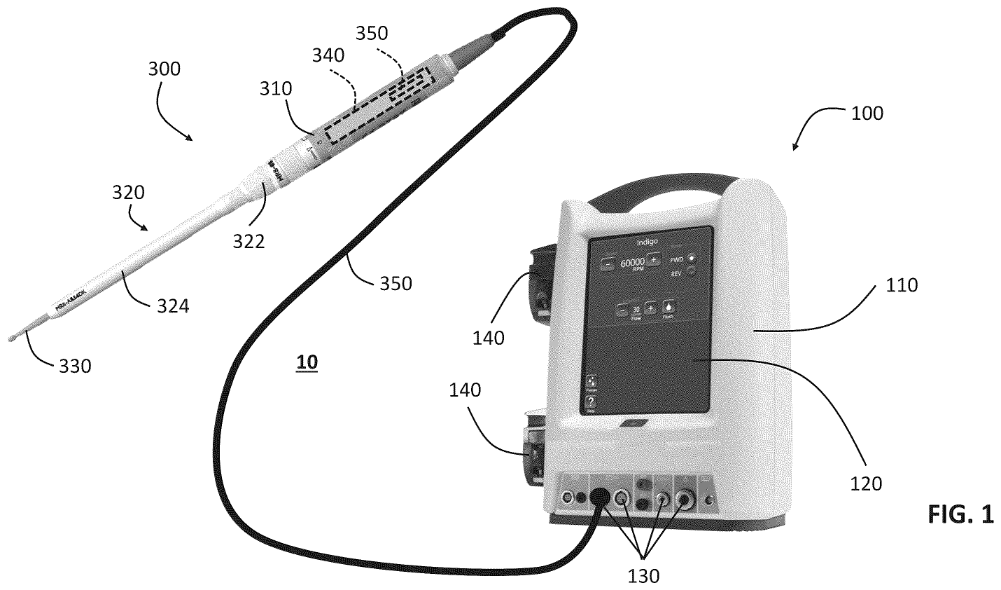

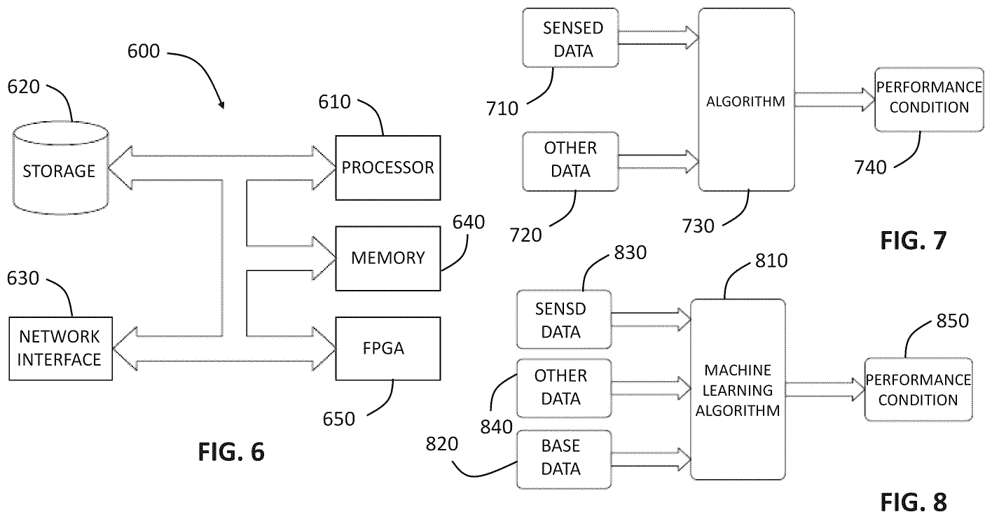

Turning to FIG. 1, a surgical system 10 provided in accordance with the present disclosure includes a console 100 and one or more surgical cutting devices 300. Console 100 may include an outer housing 110 enclosing the internal operable components of console 100, a touch screen graphical user interface (GUI) 120 to receive user input and display information to the user, a plurality of device ports 130, one or more fluid pumps 140, and/or other suitable features. One or more controllers 600 (see FIG. 6) including one or more processors and associated memory(s) are disposed within outer housing 110 and function to provide power and control signals to devices connected to console 100; to process user inputs, feedback data, and other data received at console 100; and to control the one or more fluid pumps 140. Suitable hardware and drive mechanisms as part of or in addition to controller 600 (FIG. 6) may be disposed within outer housing 110 to perform the various functions of console 100 and may include, for example, one or more central processing units (CPU's) and/or microcontroller units (MCU's), power generating and control hardware and corresponding firmware/software stored thereon, sensor circuitry, motors, pump drivers, pump controllers, etc.

The one or more surgical cutting devices 300 may define any suitable configurations for use in performing various different surgical tasks, for use in various different procedures, etc. One example of a suitable surgical cutting device, surgical cutting device 300, generally includes a handle 310, a shaft assembly 320 extending distally from handle 310 (releasably or integrally connected thereto), a cutting tool 330 extending distally from shaft assembly 320 310 (releasably or integrally connected thereto), a motor 340 disposed within handle 310 and operably coupled to cutting tool 330 to drive rotation and/or reciprocation of cutting tool 330 relative to shaft assembly 320 to cut tissue, and a cord 350 to connect motor 340 to console 100 to enable console 100 to power and control motor 340, thereby controlling cutting tool 330. In aspects, shaft assembly 320 includes a rotation collar 322 that is rotatable relative to handle 310 to advance or retract (depending upon the direction of rotation of rotation collar 322) an outer sleeve 324 of shaft assembly 320 relative to cutting tool 330 to expose more or less of cutting tool 330 at the distal end of outer sleeve 324. Motor 340 may be an electric motor, pneumatic motor, ultrasonic transducer, or other suitable motor configured to drive cutting tool 330 to rotate and/or reciprocate for cutting tissue. Console 100 is configured to drive and control motor 340 such as, for example, a speed, torque, etc. output by motor 340. In aspects, surgical cutting device 300 may include additional features such as, for example, hand control(s), navigation, articulation, etc.

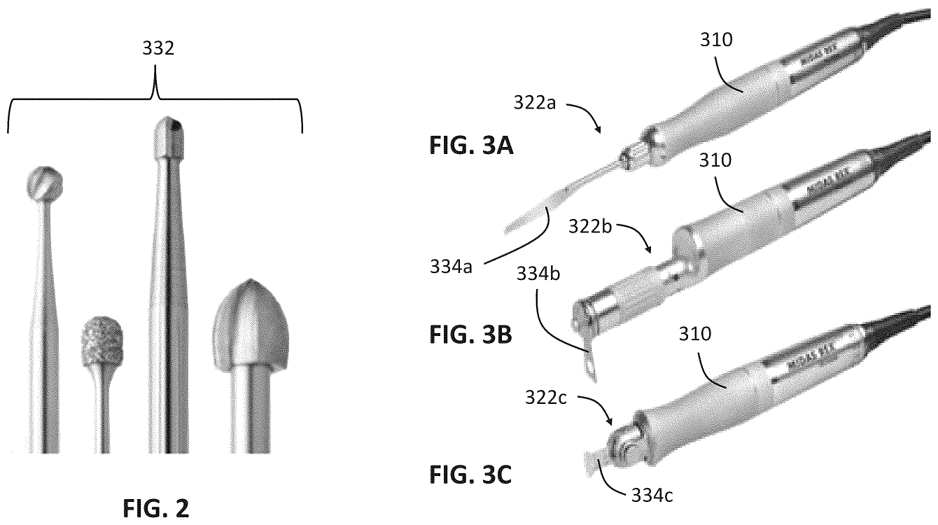

Cutting tool 330 may define any suitable configuration and may be integrated with surgical cutting device 300 or removable therefrom. More specifically, and with additional reference to FIG. 2, various different rotational cutting tools 332 may be configured for releasable attachment with surgical cutting device 300. In aspects, rotational cutting tools 332 are releasably engagable with shaft assembly 320 (which, in turn, may be releasably or integrally connected to handle 310). Alternatively, rotational cutting tools 332 may be integral with corresponding shaft assemblies 320 that are, in turn, releasably engagable with handle 310. In either configuration, surgical cutting device 300 is thus capable of being interchangeably customized with a particular rotational cutting tool 332, depending upon a particular purpose. Reciprocating cutting tools and/or cutting tools configured for both rotation and reciprocation are also contemplated.

With reference to FIGS. 3A-3C, in addition or as an alternative to rotational cutting tools 332 (FIG. 2), handle 310 may releasably or integrally connect to a shaft assembly 322a, 322b, 322c including a respective saw cutting tool 334a, 334b, 334c configured for longitudinal reciprocating motion along a longitudinal axis of the shaft assembly 322a, oscillating motion about an axis substantially parallel to a longitudinal axis of the shaft assembly 322b, or oscillating motion about an axis substantially perpendicular to a longitudinal axis of the shaft assembly 322c, respectively. Other suitable saw cutting tools are also contemplated.

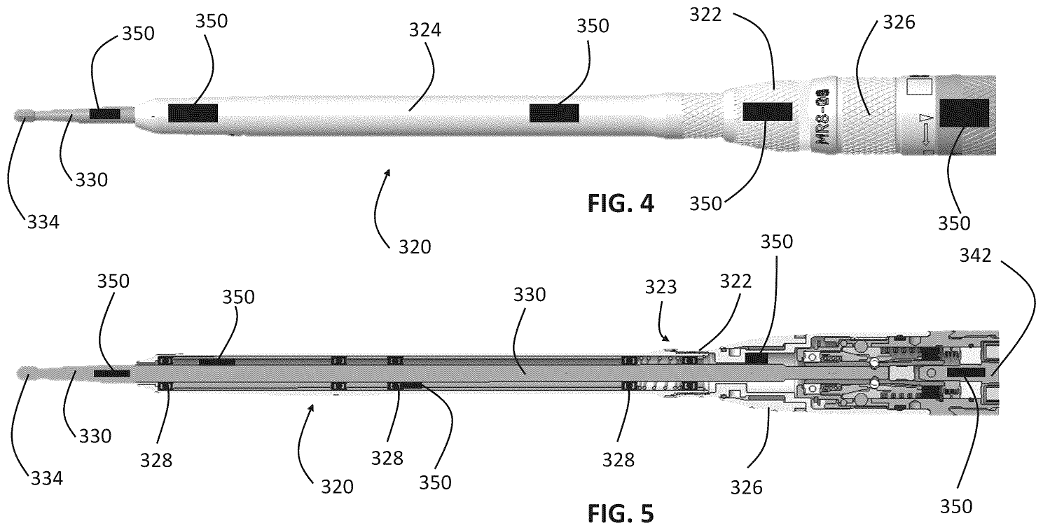

Referring to FIGS. 4 and 5, shaft assembly 320 of surgical cutting device 300 (FIG. 1) is shown including cutting tool 330 extending distally therefrom. Although detailed with respect to shaft assembly 320 and cutting tool 330, the aspects and features of the present disclosure detailed below are equally applicable for use with the other shaft assemblies and cutting tools detailed herein or any other suitable shaft assemblies and/or cutting tools.

Shaft assembly 320 includes, as noted above, rotation collar 322 and outer sleeve 324, and further includes a proximal hub 326 configured to releasably (or, in other aspects, integrally) connect shaft assembly 320 to handle 310 (FIG. 1) and a plurality of bearings 328 configured to movably support cutting tool 330 within outer sleeve 324, thus permitting rotation and/or translation of cutting tool 330 relative to outer sleeve 324 to cut tissue. Rotation collar 322 operably engages outer sleeve 324 with proximal hub 326 (and, thus, handle 310 (FIG. 1)) via a lead screw coupling 323 such that, as noted above, rotation of rotation collar 322 advances or retracts outer sleeve 324 about and relative to cutting tool 330. Although this adjustment, e.g., advancement and retraction, of outer sleeve 324 relative to cutting tool 330 is shown and described as a manual adjustment, it is also contemplated that motor 340 (FIG. 1) or a separate motor may be utilized to provide powered (and, in aspects, automatic) adjustment, e.g., advancement and retraction, of outer sleeve 324 relative to cutting tool 330.

Cutting tool 330 extends through outer sleeve 324 and may be configured for direct or indirect coupling with motor 340 (FIG. 1) to enable rotational, reciprocating, and/or other motional driving of cutting tool 330. Cutting tool 330 further includes a distal working tip 334 that extends distally from outer sleeve 324. Distal working tip 334 may define any suitable configuration such as, without limitation, those illustrated in FIG. 2.

Continuing with reference to FIGS. 4 and 5, and with additional reference to FIG. 1, surgical cutting device 300 may include one or more sensors 350 disposed at various different locations on or within one or more of the components or assemblies of surgical cutting device 300. The one or more sensors 350 may include: vibration or inertial sensors such as piezoelectric sensors, accelerometers, gyroscopes, magnetometers, or combinations thereof (e.g., to monitor vibration/motion of cutting tool 330 and/or outer sleeve 324); position or displacement sensors such as optical and/or laser sensors for obtaining displacement, position, and/or vibration data; force sensors (e.g., to monitor force, torque, and/or strain on cutting tool 330 and/or outer sleeve 324); audio sensors (e.g., to monitor noise produced by surgical cutting device 300 and/or components thereof, at the interface between cutting tool 330 and tissue, etc.); electrical property sensors (e.g., sensors configured to monitor current (such as motor current), impedance, voltage, power, chances thereof, etc.); and/or temperature sensors (e.g., to monitor the temperature of surgical cutting device 300 and/or components thereof, at the interface between cutting tool 330 and tissue, etc.). Other suitable sensors are also contemplated. In aspects, a single sensor 350 is provided. In other aspects, a plurality of sensors 350 of the same type are provided at various locations on or within surgical cutting device 300. In still other aspects, one or more sensors 350 of a first type and one or more sensors 350 of a second, different type are provided at the same and/or different locations on or within surgical cutting device 300.

With respect to the location(s) of sensor(s) 350 on or within surgical cutting device 300, a sensor 350 may be disposed, for example and as shown in FIGS. 4 and 5: on or within rotation collar 322; on or within outer sleeve 324 (towards the proximal end, distal end, and/or at an intermediate location of outer sleeve 324); between outer sleeve 324 and cutting tool 330; on or within proximal hub 326; coupled to or incorporated within a bearing 328; on or within cutting tool 330 (within outer sleeve 324 and/or on or within exposed portion of cutting tool 330; towards the proximal end, distal end, and/or at an intermediate location of cutting tool 330); on or within a drive rotor 342 of motor 340 (FIG. 1) that is driven by motor 340 (FIG. 1) to, in turn, drive movement of cutting tool 330; and/or associated with motor 340 (FIG. 1) and/or the electrical inputs thereto (whether disposed on or within motor 340 (FIG. 1) or remote therefrom such as, for example, within console 100 (FIG. 1)). A sensor 350 may be provided at any other suitable location. In particular, a sensor 350 may be provided at a location that experiences detectable changes in the property to be sensed depending upon the performance condition of surgical cutting device 300. For example, temperature sensors, vibration sensors, and/or force sensors may be disposed at respective locations that tend to heat up, vibrate more, and/or experience increased forces when surgical cutting device 300 is operating in an unstable condition as compared to a normal condition.

Suitable electrical wires, electrically conductive structures, electrical traces, contacts, wireless connection interfaces, combinations thereof, etc. (not explicitly shown) are provide on or within surgical cutting device 300 (and/or the components thereof) to electrically couple the one or more sensors 350 with console 100.

Regardless of the particular type and/or location of the one or more sensors 350, the one or more sensors 350 are configured to provide data indicative of one or more properties of surgical cutting device 300 that, alone or in combination with feedback from additional sensors 350, other sensors associated with or separate from surgical cutting device 300, data input by a user, input read/received from components of surgical cutting device 300 or other devices, and/or other data, enables determination of a performance condition of surgical cutting device 300. The performance condition of surgical cutting device 300 may include, for example, stability and/or efficiency. With respect to stability, the sensor data and, in aspects, additional data, may be utilized to determine a stability of surgical cutting device 300 during use (and, in aspects, in real time). The stability may be a binary output, e.g., whether the surgical cutting device 300 is operating in a stable manner or an unstable manner. Alternatively, the stability may be provided as a level of stability, for example, on a numerical scale (e.g., stability on a scale of 1-10 or 1-100) or on a symbolic scale (e.g., stability indicated as green, yellow, or red). Sensor data indicative of stability of surgical cutting device 300 that may be utilized to determine the stability performance condition includes, for example and without limitation: vibration or motion data; force, torque, and/or strain data; audio data; and/or temperature data.

With respect to efficiency, the sensor data and, in aspects, additional data, may be utilized to determine an efficiency of surgical cutting device 300 during use (and, in aspects, in real time). The efficiency may be a binary output, e.g., whether the surgical cutting device 300 is operating in an efficient manner or an inefficient manner. Alternatively, the stability may be provided as a level of efficiency, for example, on a numerical scale (e.g., efficiency on a scale of 1-10 or 1-100) or on a symbolic scale (e.g., efficiency indicated as green, yellow, or red). Sensor data indicative of efficiency of surgical cutting device 300 that may be utilized to determine the efficiency performance condition includes, for example and without limitation: vibration or motion data; force, torque, and/or strain data; and/or electrical properties.

Other sensors associated with or separate from surgical cutting device 300 that may provide data suitable for use in determining the performance condition of surgical cutting device 300 include, for example and without limitation, an image sensor to enable real time imaging, e.g., video imaging, thermal imaging, ultrasound imaging, etc., of a field of view including at least cutting tool 330 and/or tissue being cut with cutting tool 330; an electrical impedance and/or other electrical characteristic sensor, e.g., to measure tissue electrical conductivity (and/or other electrical properties) of tissue being cut; a force/pressure sensor, e.g., to measure force or pressure applied to tissue being cut); and/or other suitable sensors. Such sensors may enable, for example, determination of properties of the tissue being cut and/or to enable determination of the type of tissue being cut. Determination of properties of tissue being cut include tissue category (soft tissue or hard tissue), tissue thickness, tissue density, tissue condition (healthy or diseased), transitions between tissues (e.g., tissue layers, between types of tissue, etc. entry or exit to/from anatomical cavities), etc. Determination of the type of tissue being cut includes determination of whether the tissue is, for example, bone, cartilage, muscle, organ, etc.

Referring still to FIGS. 1, 4, and 5, data regarding properties of the tissue being cut and/or the type of tissue being cut may be utilized together with the data from the one or more sensors 350 to determine the performance condition of surgical cutting device 300 during use. More specifically, because surgical cutting device 300 may exhibit different properties when cutting tissue having different properties and/or tissues of different type (such as, for example, where surgical cutting device 300 generates more heat and vibration when cutting bone or harder tissue as compared to cartilage or softer tissue, or where a spike in motor current occurs in response to transitioning from cutting soft tissue to cutting hard tissue), data regarding the properties and/or types of tissue being cut can be utilized to contextualize the data provided by sensors 350. For example, temperatures, amplitudes of vibration, and/or motor currents, or changes thereof, may be typical for cutting one type of tissue or tissue with certain properties; however, the same temperatures, amplitudes of vibration, and/or motor currents, or changes thereof, may indicate instability and/or inefficiency when exhibited during cutting of another type of tissue or tissue with other properties.

Other data which may be utilized to facilitate determination of the performance condition of surgical cutting device 300 during use may include data input by a user, input read/received from components or devices, and/or other data. This data may include data relating to surgical cutting device 300 and/or components thereof (e.g., the attached shaft assembly 320 and/or cutting tool 330) including, for example and without limitation: device/component ID; device/component type; device/component lot number; device/component manufacture date; surgeon and/or hospital data; patient data; procedure data; etc. Because surgical cutting device 300 may exhibit different properties when cutting tissue depending upon the type, age, and/or configuration of surgical cutting device 300 (and/or the components thereof), the technique utilized, the approach taken, the experience of the surgeon, the type of procedure being performed, and/or the condition and/or anatomy of the patient, such data can be utilized to contextualize the data provided by sensors 350.

For example, forces, strains, and/or torques, or changes thereof, encountered during use of surgical cutting device 300 may be typical for one procedure or technique; however, the same forces, strains, and/or torques, or changes thereof, may indicate instability and/or inefficiency when exhibited during cutting of tissue in another procedure or using another technique. As another example, temperatures, amplitudes of vibration, and/or motor currents, or changes thereof, may be typical when one type and/or age of cutting tool 330, shaft assembly 320, and/or handle 310 is utilized; however, the same temperatures, amplitudes of vibration, and/or motor currents, or changes thereof, may indicate instability and/or inefficiency when exhibited during use of another type and/or age of cutting tool 330, shaft assembly 320, and/or handle 310.

In aspects, in order to read/write at least some of the other data noted above, some or all components of surgical cutting device 300, e.g., handle 310, shaft assembly 320, and/or cutting tool 330, include RFID or other suitable communication chips (not explicitly shown) having memories storing data. For example, handle 310, shaft assembly 320, and/or cutting tool 330 may include memories, e.g., read-only memories, storing identifying data that can be read by console 100 (e.g., unique ID, device/component type, lot number, manufacture date, configuration data, features, components, and/or settings). Handle 310, shaft assembly 320, and/or cutting tool 330 may additionally or alternatively include memories, e.g., read/write memories, that can be read and/or written to by console 100 storing, for example, a use count, a sterilization count, usage data, an event/error log, use and/or operational flags, etc. Data transmitted to/from surgical cutting device 300 (and/or components thereof) may be transmitted via a wired or wireless network or in any other suitable manner for storage in a remote server (including cloud servers) to enable managing and tracking such information. Further, rather than on-board memories, surgical cutting device 300 (and/or components thereof) may include barcodes or other identifiers to enable association of data thereof with the surgical cutting device 300 (and/or components thereof), thereby enabling management and tracking at the remote server. In addition, additional data may include data from a robotic surgical system, navigation system, and/or other system(s) associated with use of surgical cutting device 300.

Turning to FIG. 6, a controller 600 of console 100 (FIG. 1) is detailed. Although controller 600 is detailed as part of console 100 (FIG. 1), controller 600 may alternatively be incorporated into surgical cutting device 300 (FIG. 1), distributed across console 100 and surgical cutting device 300, or distributed across multiple devices including cloud server(s) and/or remote device(s) connected via a network or other communication link. Controller 600 includes a processor 610 connected to a computer-readable storage medium or a memory 620 which may be a volatile type memory, e.g., RAM, or a non-volatile type memory, e.g., flash media, disk media, etc. In aspects, processor 610 may be, without limitation, a digital signal processor, a microprocessor, an ASIC, a graphics processing unit (GPU), field-programmable gate array (FPGA), or a central processing unit (CPU). In aspects, memory 620 can be random access memory, read-only memory, magnetic disk memory, solid state memory, optical disc memory, and/or another type of memory. In aspects, memory 620 can be separate from controller 600 and can communicate with processor 610 through communication buses of a circuit board and/or through communication cables such as serial ATA cables or other types of cables. Memory 620 includes computer-readable instructions that are executable by processor 610 to operate controller 600. In aspects, controller 600 includes a network interface 630 to communicate with other computers or a server. In aspects, a storage device 640 may be used for storing data. In embodiments, controller 600 may include one or more FPGAs 650. FPGA 650 may be used for performing computations and/or executing algorithms including machine learning algorithms.

Memory 620 stores suitable instructions, to be executed by processor 610, for receiving the sensed data, e.g., sensed data from sensors 350 (FIGS. 4 and 5), and any other data; accessing storage device 640 of controller 600; determining the performance condition of surgical cutting device 300 (FIG. 1); and, based upon the determined performance condition, adjusting operational settings (thus providing automatic adaptive control) and/or recommending changes (thus enabling manual adaptive control).

Determining the performance condition of surgical cutting device 300 (FIG. 1) may initially include processing the received data, e.g., data from sensors 350 (FIGS. 4 and 5). This processing may include, for example, evaluating the sensor data over time, computing statistics (averages, maximums, minimums, etc.), determining rates of change (direction and amplitude), identifying steady-state conditions and deviations from the steady-state conditions, etc.

With additional reference to FIG. 7, the sensed data 710 (whether processed data or the original data, in aspects where initial processing is not performed), together with other data 720, is input into one or more algorithms 730, e.g., including one or more thresholds stored in one or more look-up tables of storage device 640, to determine the performance condition(s) 740. The algorithm(s) 730 and, more specifically, the applicable thresholds thereof, may vary depending upon time data, different processed data from the same sensor(s) 350, data from different sensors 350, and/or other data 720. For example, an adverse performance condition (e.g., instability and/or inefficiency) may be determined where sensed data exceeds a first threshold for a first amount of time or where the sensed data does not exceed the first threshold but exceeds a second, lower threshold for a second, longer amount of time. As another example, an adverse performance condition (e.g., instability and/or inefficiency) may be determined where first sensed data exceeds a first threshold without consideration of other sensed data or where the first sensed data does not exceed the first threshold but exceeds a second, lower threshold and second sensed data also exceeds a threshold. As still another example, thresholds need not be universal but may be adjusted depending upon, for example, the other data obtained that may help contextualize the sensor data, as detailed above.

As noted above, the determination of a performance condition 740, e.g., stability and/or efficiency, may be a binary output or a scaled (numerical or symbolic) output. In configurations where a scaled output is provided, different thresholds may be utilized to determine the performance condition 740 on the corresponding scale. Regardless of the form of the determination, once the determination is made, controller 600 provides instructions and/or an output based upon the determined performance condition 740, as detailed below.

Referring to FIGS. 6 and 8, in aspects, one or more machine learning algorithms 810 are utilized by controller 600 to determine the performance condition. The machine learning algorithm(s) 810 may be trained on and learn from base data 820, e.g., experimental data and/or data from previous procedures initially input into one or more machine learning algorithms 810; the sensed data 830, e.g., sensed data from sensor(s) 350 (FIGS. 4 and 5); and/or other data 840 in order to enable the machine learning algorithm(s) 810 to determine the performance condition 850. In aspects, training the machine learning algorithm(s) 810 may be performed by a computing device outside of console 100 (FIG. 1) and the resulting algorithm may be communicated to controller 600. Further, training may be initially performed and the machine learning algorithm(s) 810 thereafter locked, or machine learning algorithm(s) 810 may be trained and updated based on data obtained during use, e.g., continuously or discretely in accordance with scheduled updates.

The determination of a performance condition 850, e.g., stability and/or efficiency, as noted above, may be a binary output or a scaled (numerical or symbolic) output. The output, e.g., binary or scaled, may dictate the type of machine learning algorithm(s) 810 utilized. For example, classification machine learning techniques may be utilized where a binary output or scaled output of a relatively few selections is utilized. On the other hand, regression machine learning techniques may be utilized where a scaled output of a relatively large number of selections is utilized (e.g., 1-100). The machine learning algorithm(s) 810 may implement one or more of: supervised learning, semi-supervised learning, unsupervised learning, reinforcement learning, association rule learning, decision tree learning, anomaly detection, feature learning, computer vision, etc., and may be modeled as one or more of a neural network, Bayesian network, support vector machine, genetic algorithm, etc. Once the determination is made, controller 600 provides instructions and/or an output based upon the determined performance condition 850, as detailed below.

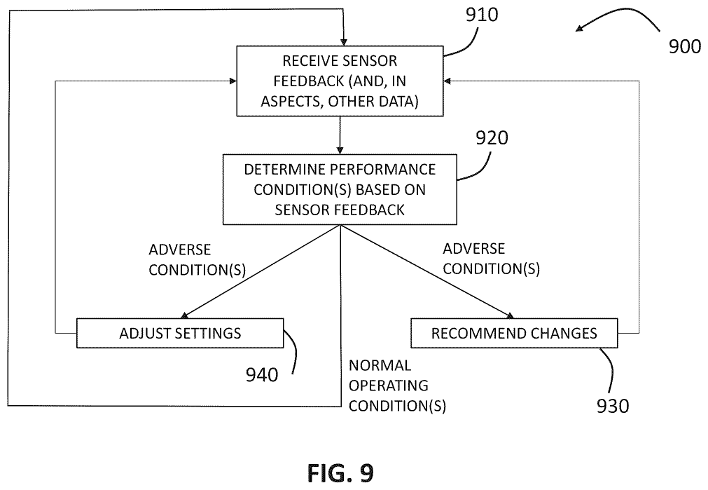

Turning to FIG. 9, a method 900 in accordance with the present disclosure is shown. As noted above, during use, sensor feedback and/or other data is received, as indicated at 910. Based upon this sensor feedback and/or other data, as indicated at 920, one or more performance conditions are determined. The determination of the one or more performance conditions may be made continuously, periodically, or in any other suitable manner. With respect to a binary output of the determination of the one or more performance conditions, for example, if it is determined that an adverse performance condition exists, e.g., instability and/or inefficiency, the method proceeds to 930 and/or 940. At 930, a recommended change is output, e.g., controller 600 (FIG. 6) provides instructions to output a recommended change in the form of, for example, a visual output, e.g., graphics and/or text on GUI 120 of console 100 (see FIG. 1), and/or an audible output, e.g., from a speaker associated with console 100 (FIG. 1). The recommended change may direct a user to take one or more corrective actions to remedy the adverse performance condition(s), e.g., instability and/or inefficiency. The recommended corrective actions may include, for example, recommending: changing technique, changing ergonomic position to better balance and/or support the surgical cutting device 300 (FIG. 1), use of a lower speed, reducing pressure/force, choking up on the surgical cutting device 300 (FIG. 1), reducing exposure of the cutting tool 330 (FIG. 1; e.g., via rotating rotation collar 322 to advance or retract outer sleeve 324 of shaft assembly 320 relative to cutting tool 330), changing tool type and/or size, etc. With respect to a scaled output, the number and/or type of corrective actions recommended may vary depending upon the scaled output.

At 940, alternatively or additionally, if it is determined that an adverse performance condition exists, e.g., instability and/or inefficiency, controller 600 (FIG. 6) provides instructions to automatically adjust settings of console 100 and/or surgical cutting device 300 (see FIG. 1) based on the determined adverse performance condition. For example, controller 600 (FIG. 6) may provide instructions to control motor 340 of surgical cutting device 300 (see FIG. 1) to automatically reduce motor speed, reduce motor torque, advance or retract outer sleeve 324 of shaft assembly 320 relative to cutting tool 330 (in aspects where such adjustment is automated), etc. With respect to a scaled output, the severity and/or type of corrective actions taken may vary depending upon the scaled output.

In aspects, in response to detection of an adverse performance condition (or adverse performance condition above a threshold), the automatic adjustment may include a safety shutdown preventing operation or a safety pause preventing operation for a determined amount of time and/or until the adverse performance condition ceases. In robotic implementations, this may additionally or alternatively include moving the instrument out of the surgical site or to a safe location within the surgical site.

Further, in aspects where the adverse performance condition is an overheating or thermal condition (as sensed by a temperature sensor or thermal camera, for example), the automatic adjustment may include increasing irrigation flow (where such is provided for use with surgical cutting device 300 (FIG. 1)) to increase cooling of the surgical cutting device 300 (FIG. 1) and recovery from the adverse performance condition.

After a change is recommended at 930 and/or settings are adjusted at 940, or where it is determined that an adverse performance condition does not exist (that is, where normal performance conditions are detected), the method reverts to 910 to evaluate new data and again determine, as detailed above, whether an adverse performance condition exists and, if so, what action to take in response thereto.

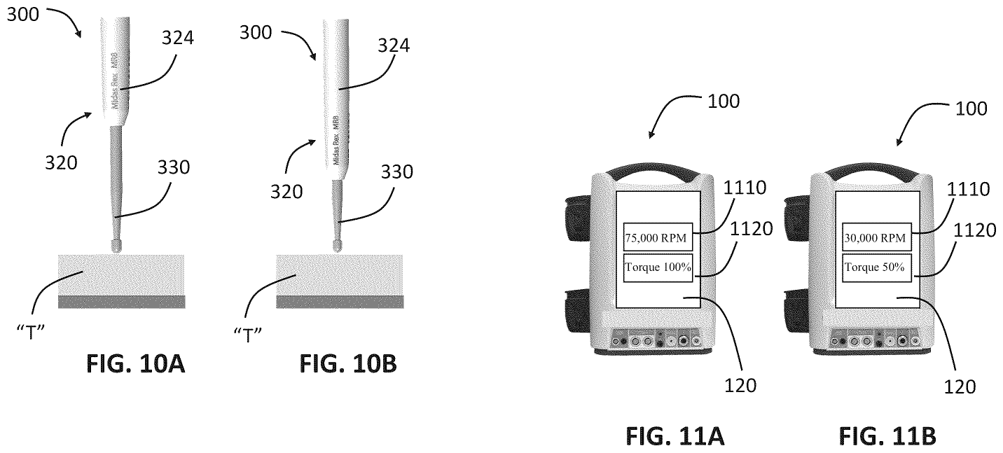

With reference to FIGS. 10A and 10B, a distal portion of surgical cutting device 300 is shown in use cutting tissue “T” with outer sleeve 324 of shaft assembly 320 disposed in retracted and extended positions, respectively, relative to cutting tool 330 to expose more or less of cutting tool 330 at the distal end of outer sleeve 324. In the retracted position (FIG. 10A), wherein more of cutting tool 330 is exposed at the distal end of outer sleeve 324, cutting tool 330 is less constrained and, thus, movement of cutting tool 330 is less dampened and/or the cutting tool 330 is relatively less stiff. This may enable increased cutting performance during use in a normal performance condition. However, in this retracted position (FIG. 10A), surgical cutting device 300 may be more prone to instability and/or inefficiency when operating in an adverse performance condition. Thus, depending upon the performance condition(s) determined, outer sleeve 324 may be automatically moved or a recommendation to move outer sleeve 324 may be provided to move outer sleeve 324 from the retracted position (FIG. 10A) to the extended position (FIG. 10B). In the extended position (FIG. 10B), less of cutting tool 330 is exposed at the distal end of outer sleeve 324 such that cutting tool 330 is more constrained and, thus, movement of cutting tool 330 is dampened and vibrations of cutting tool 330 are reduced. Additionally or alternatively, a stiffness of cutting tool 330 is increased to thereby constrain motion of cutting tool 330. Thus, greater stability and/or efficiency are achieved. In configuration where the performance condition is determined according to a scale, the extended position, e.g., the extent to which outer sleeve 324 is moved distally about cutting tool 330, may be determined based upon the scaled performance condition, e.g., where outer sleeve 324 is advanced further distally about cutting tool 330 when more severe instability is detected as compared to less severe instability. Regardless of the particular implementation, the present disclosure enables live tuning (or recommendation of the same) of surgical cutting device 300 based on system feedback which, in some situations, may depart from the direction and/or conventional wisdom of the operator, thus providing adaptive control above and beyond that which may be provided manually.

Referring to FIGS. 11A and 11B, console 100 is shown wherein GUI 120 is displaying initial operating settings during use for speed (RPM's) 1110 and torque (% of a pre-determined torque reference value) 1120 and adjusted operating settings during use for speed 1110 and torque 1120, respectively. Depending upon the performance condition(s) determined, controller 600 (FIG. 6) may automatically adjust the speed 1110 and/or torque 1120 settings of motor 340 (FIG. 1) in order to improve stability and/or efficiency and enable safe continued use in response to detection of an adverse performance condition. More specifically, reducing speed and/or torque may reduce vibrations, temperature hot spots, excess forces, etc., thus increasing stability and/or efficiency. The amount of speed and/or torque adjustment (e.g., decrease) may depend upon, for example the level of instability and/or inefficiency detected (e.g., where a scaled performance condition is determined).

While several aspects of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical system having adaptive control, comprising:
a surgical cutting device including a cutting tool and a motor configured to drive movement of the cutting tool;
at least one sensor configured to produce sensor data indicative of at least one property of the surgical cutting device during use; and
a controller configured to receive the sensor data and determine a performance condition of the surgical cutting device based at least on the sensor data, the controller further configured, where the determined performance condition is an adverse performance condition, to at least one of: adjust settings of the surgical cutting device or recommend a change relating to use of the surgical cutting device.

2. The surgical system according to claim 1, wherein the controller is further configured to receive other data and to determine the performance condition based at least on the sensor data and the other data.

3. The surgical system according to claim 2, wherein the other data includes at least one of: identifying data, patient data, procedure data, robotic system data, or navigation data.

4. The surgical system according to claim 1, wherein the determined performance condition includes at least one of a stability condition or an efficiency condition.

5. The surgical system according to claim 1, wherein the determined performance condition is a binary determination.

6. The surgical system according to claim 1, wherein the determined performance condition is a scaled determination.

7. The surgical system according to claim 1, wherein the controller is configured, where the determined performance condition is an adverse performance condition, to adjust settings of the surgical cutting device by adjusting at least one of: a speed of the motor; a torque of the motor; or a performance impacting component of the surgical cutting device.

8. The surgical system according to claim 1, wherein the controller is configured, where the determined performance condition is an adverse performance condition, to recommend a change relating to use of the surgical cutting device by recommending a manual change in a performance impacting component of the surgical cutting device.

9. The surgical system according to claim 1, wherein the at least one sensor includes at least one of: a vibration sensor, an audio sensor, a force sensor, a torque sensor, a temperature sensor, an optical sensor, or a motor electrical property sensor.

10. The surgical system according to claim 1, wherein the surgical cutting device further includes a handle housing the motor therein and a shaft assembly coupled to the handle and including an outer sleeve, wherein the cutting tool extends through the outer sleeve of the shaft assembly.

11. The surgical system according to claim 10, wherein the at least one sensor is disposed on or within at least one of: the handle, the outer sleeve, or the cutting tool.

12. The surgical system according to claim 1, wherein the controller is configured, in real time, to determine the performance condition and, where the determined performance condition is an adverse performance condition, at least one of: adjust the settings of the surgical cutting device or recommend the change relating to use of the surgical cutting device.

13. The surgical system according to claim 1, further comprising a console configured to supply power and control signals to the surgical cutting device, wherein the controller is disposed within the console.

14. The surgical system according to claim 1, wherein the controller is configured to implement at least one machine learning algorithm to determine the performance condition.

15. A method of adaptive control of a surgical system, comprising:
driving a motor to move a cutting tool of a surgical cutting device to cut tissue;
monitoring, during the cutting of the tissue, sensor data indicative of at least one property of the surgical cutting device;
determining a performance condition of the surgical cutting device based at least on the sensor data; and
where the determined performance condition is an adverse performance condition, at least one of: adjusting settings of the surgical cutting device or recommending a change relating to use of the surgical cutting device.

16. The method according to claim 15, further comprising:
receiving other data including at least one of: identifying data, patient data, or procedure data,
wherein the performance condition is determined based at least on the sensor data and the other data.

17. The method according to claim 15, wherein the determined performance condition includes at least one of a stability condition or an efficiency condition.

18. The method according to claim 15, wherein, where the determined performance condition is an adverse performance condition, the settings of the surgical cutting device are adjusted by adjusting at least one of: a speed of the motor; a torque of the motor; or a performance impacting component of the surgical cutting device.

19. The method according to claim 15, wherein, where the determined performance condition is an adverse performance condition, a change relating to use of the surgical cutting device is recommended by recommending a manual change in a performance impacting component of the surgical cutting device.

20. The method according to claim 15, wherein the sensor data includes at least one of: vibration data, audio data, torque data, optical data, force data, temperature data, or motor electrical property data.
